(19) United States
(12) Patent Application Publication
(10) Pub. No.: US 2014/0155744 A1
(43) Pub. Date: Jun. 5, 2014

(54) Power Injection Catheter Assembly And Method Of Using Same

(71) Applicant: Preston Pameijer, Bloomfield, IN (US)

(72) Inventor: Preston Pameijer, Bloomfield, IN (US)

(21) Appl. No.: 13/690,363

(22) Filed: Nov. 30, 2012

(51) Int. Cl. A61M 5/00

(52) U.S. Cl. CPC A61M 5/007
USPC 600/435

(57) ABSTRACT

A power injection catheter assembly includes a power injection catheter having an elongate tubular body. The elongate tubular body has a proximal end and a distal end and defines a power injection lumen. A manifold is attached at the proximal end of the elongate tubular body and defines a first power injection channel extending from a first power injection port of the manifold to the power injection lumen, and a second power injection channel extending from a second power injection port of the manifold to the power injection lumen. A first power injection extension tube has a proximal end defining a first power injection hub and a distal end attached to the manifold at the first power injection port. A second power injection extension tube has a proximal end defining a second power injection hub and a distal end attached to the manifold at the second power injection port.

### TECHNICAL FIELD

The present disclosure relates generally to a power injection catheter assembly, and more particularly to a power injection catheter assembly having multiple power injection hubs fluidly supplying a common power injection lumen.

### BACKGROUND

Catheters are well known and used in a variety of different medical procedures, including the injection of contrast media into the bloodstream of a patient. The delivery of contrast media is most effective and efficient if the contrast media is delivered at specific flow rates. For example, a power injector may be configured to push the contrast media into and through the delivery catheter at controlled volumes and flow rates, which may be about two to three cubic centimeters capacity per second or higher. Conventional catheters may be unable to withstand the high flow rates required for power injection. As such, catheters particularly configured for power injection may exhibit improved catheter wall strength capable of withstanding high flow applications.

Depending on the particular application, catheters having multiple lumens are often selected to avoid repeated vascular access. However, utilizing a catheter having walls increased in geometry and/or durometer for use in the power injection applications described above may increase the stiffness of the catheter and may limit the number of catheter lumens. As should be appreciated, catheters having reduced size and increased flexibility are often desirable to ease navigation by the clinician and improve patient comfort. Thus, a typical power injection catheter, having strengthened catheter walls, will often have only one power injection lumen. If multiple power injection lumens are provided, there is usually a sacrifice relative to size and stiffness of the catheter.

An exemplary power injection catheter is shown in U.S. Patent Application Publication 2006/0149214 to Breiter et al. According to a particular embodiment, as shown in FIG. 14, a power injection catheter may include a single power injection lumen having two extension tubes in communication with the lumen via a manifold, with a first of the extension tubes being configured for low flow delivery and a second of the extension tubes being configured for high flow delivery. A tubing clamp may be used to selectively switch between a high flow application and a low flow application. Although the power injection catheter of Breiter et al. may be suitable for some applications, there is a continuing need for versatility in the field.

The present disclosure is directed toward one or more of the problems set forth above.

### SUMMARY OF THE DISCLOSURE

In one aspect, a power injection catheter assembly includes a power injection catheter having an elongate tubular body. The elongate tubular body has a proximal end and a distal end and defines a power injection lumen. A manifold is attached at the proximal end of the elongate tubular body and defines a first power injection channel extending from a first power injection port of the manifold to the power injection lumen, and a second power injection channel extending from a second power injection port of the manifold to the power injection lumen. A first power injection extension tube has a proximal end defining a first power injection hub and a distal end attached to the manifold at the first power injection port. A second power injection extension tube has a proximal end defining a second power injection hub and a distal end attached to the manifold at the second power injection port.

In another aspect, a method of power injecting fluids using the power injection catheter assembly includes steps of power injecting a first power injectable fluid through the first power injection extension tube and into the manifold and power injecting a second power injectable fluid through the second power injection extension tube and into the manifold. The first power injectable fluid and the second power injectable fluid are directed toward the power injection lumen using the manifold. The first and second power injectable fluids are then power injected through the power injection lumen and into a patient vessel.

### BRIEF DESCRIPTION OF THE DRAWINGS

FIG. 1 is a partially sectioned side diagrammatic view of a single lumen power injection catheter assembly, according to one embodiment of the present disclosure;

FIG. 2 is a partially sectioned side diagrammatic view of a portion of a single lumen power injection catheter assembly, according to another embodiment of the present disclosure;

FIG. 3 is a partially sectioned side diagrammatic view of a portion of a dual lumen power injection catheter assembly, according to another embodiment of the present disclosure; and

FIG. 4 is a partially sectioned side diagrammatic view of a patient vessel at one stage of a percutaneous endovascular procedure, according to another aspect of the present disclosure.

### DETAILED DESCRIPTION

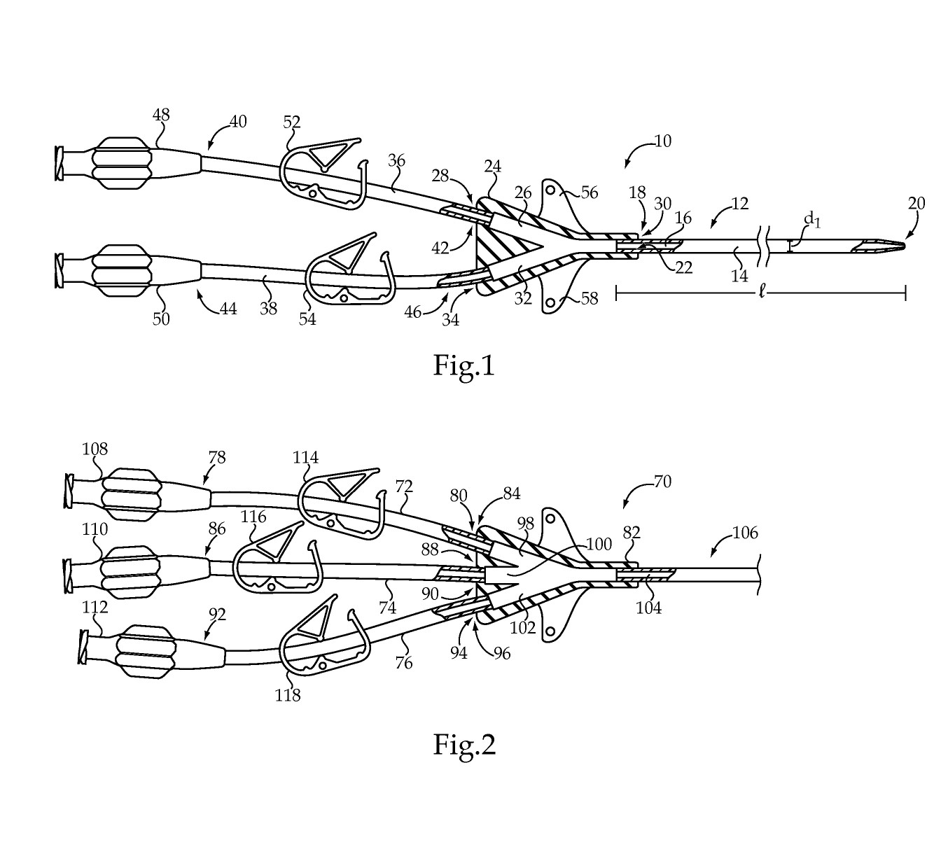

Referring to FIG. 1, there is shown a power injection catheter assembly 10 according to one embodiment of the present disclosure. Although the power injection catheter assembly 10 may be used in a variety of percutaneous endovascular procedures, a specific use of the power injection catheter assembly 10 may include the power injection of a contrast media. The power injection catheter assembly 10 may generally include a power injection catheter 12 having an elongate tubular body 14. The elongate tubular body 14 has a length l and defines a power injection lumen 16 extending from an open proximal end 18 to an open distal end 20, which may include a distal taper. In the present disclosure, “proximal” will be used to refer to the end of a component or feature that is closest to a clinician, while “distal” is used to refer to a component or feature that is farthest away from the clinician. Such meanings are consistent with conventional use of the terms and, as such, should be understood by those skilled in the art.

The power injection catheter 12 of the present disclosure is configured as a high strength catheter capable of withstanding the high flow rates and pressures of power injection. For example, the elongate tubular body 14 may be configured to withstand flow rates of at least about 1 cubic centimeter capacity per second and pressures of at least about 100 pounds per square inch. In particular, walls 22 defining the power injection lumen 16 are configured for power injection of a power injectable fluid, as described in greater detail below. Although materials and dimensions may vary depending on the particular application, the elongate tubular body 14, according to some embodiments may have an outer diameter d₁ of less than about 12 French. As should be appreciated, the diameter d₁ may be orders of magnitude smaller than the length l.

The elongate tubular body 14 may be made from any common medical tube material, such as, for example, a plastic, rubber, silicone, or Teflon® material, and may exhibit both stiffness, or firmness, and flexibility. According to some embodiments, the elongate tubular body 14 may be made from a thermoplastic elastomer, such as polyurethane. As should be appreciated, the catheter walls 22 may have an increased geometry and/or durometer, as compared to conventional catheters, to facilitate use in high flow, or power injection, applications. Alternatively and/or additionally, the elongate tubular body 14 may include a reinforcement to increase performance in high power applications. According to some embodiments, the power injection catheter 12 may be a peripherally inserted central catheter (PICC). However, the power injection catheter assembly 10, including alternative embodiments, disclosed herein may be applicable to a variety of procedures for establishing vascular or other access to a body of a patient.

The power injection catheter assembly 10 also includes a manifold 24 attached at the open proximal end 18 of the elongate tubular body 14. According to the exemplary embodiment of FIG. 1, the manifold 24 defines a first power injection channel 26 extending from a first power injection port 28, or opening, of the manifold 24 to an open distal end 30, which is in fluid communication with the power injection lumen 16 of the power injection catheter 12. The manifold 24 also defines a second power injection channel 32 extending from a second power injection port 34 of the manifold 24 to the open distal end 30. According to some embodiments, the manifold 24 may be a relatively rigid component made from a medical grade material, such as, for example, polyurethane, and may be capable of withstanding the high flow rates and pressures of power injection. Those skilled in the art should appreciate that fluid flow testing may be performed on medical components, including catheters, hubs, manifolds, etc., to confirm suitability for power injection.

The power injection catheter assembly 10 of FIG. 1 also includes two power injection extension tubes 36 and 38. A first power injection extension tube 36 has a proximal end 40 defining a first power injection hub and a distal end 42 attached to the manifold 24 at the first power injection port 28. A second power injection extension tube 38 has a proximal end 44 defining a second power injection hub and a distal end 46 attached to the manifold 24 at the second power injection port 34. As shown, the power injection extension tubes 36 and 38 extend in the proximal direction from the manifold 24 and are both in fluid communication with the power injection lumen 16 through the power injection channels 26 and 32 of the manifold 24. As shown, the first power injection channel 26 of the manifold 24 and the second power injection channel 32 of the manifold 24 converge within the manifold 24 to combine fluid flow from the first power injection extension tube 36 and the second power injection extension tube 38.

A first connecting device 48 is provided at the proximal end 40 of the first power injection extension tube 36 to define the first power injection hub, while a second connecting device 50 is provided at the proximal end 44 of the second power injection extension tube 38 to define the second power injection hub. According to some embodiments, one or both of the connecting devices 48 and 50 may be configured for engagement with a treatment device, such as, for example, a power injector. Clamps 52 and 54 may also be provided for selectively closing off fluid flow through a respective one of the power injection extension tubes 36 and 38 in a known manner. It should be appreciated that the power injection catheter assembly 10 may include additional devices, components, and/or features, as necessary, depending on the particular procedure being performed. For example, the manifold 24 may include a pair of suture wings 56 and 58 for securing a position of the manifold 24 relative to the patient. It should also be appreciated that connections or attachments between components of the power injection catheter assembly 10 may be made using attachment means known to those skilled in the art.

According to another exemplary embodiment of the present disclosure, a power injection catheter assembly 70 may include three power injection extension tubes 72, 74, and 76. In particular, a first power injection extension tube 72 has a proximal end 78 defining a first power injection hub and a distal end 80 attached to a manifold 82 at a first power injection port 84 of the manifold 82. A second power injection extension tube 74 has a proximal end 86 defining a second power injection hub and a distal end 88 attached to the manifold 82 at a second power injection port 90 of the manifold 82. A third power injection extension tube 76 has a proximal end 92 defining a third power injection hub and a distal end 94 attached to the manifold 82 at a third power injection port 96 of the manifold 82. According to the exemplary embodiment of FIG. 2, first, second, and third power injection channels 98, 100, and 102 defined by the manifold 82 all converge within the manifold 82 to combine fluid flow from the power injection extension tubes 72, 74, and 76 and direct the combined fluid flow into a power injection lumen 104 of a power injection catheter 106. The power injection catheter assembly 70 of FIG. 2 may also include connectors 108, 110, and 112 and clamps 114, 116, and 118, similar to those described above with reference to FIG. 1. Additional power injection extension tubes and corresponding manifold channels are also contemplated for power injecting a plurality of liquid through a common catheter lumen rated for power injection.

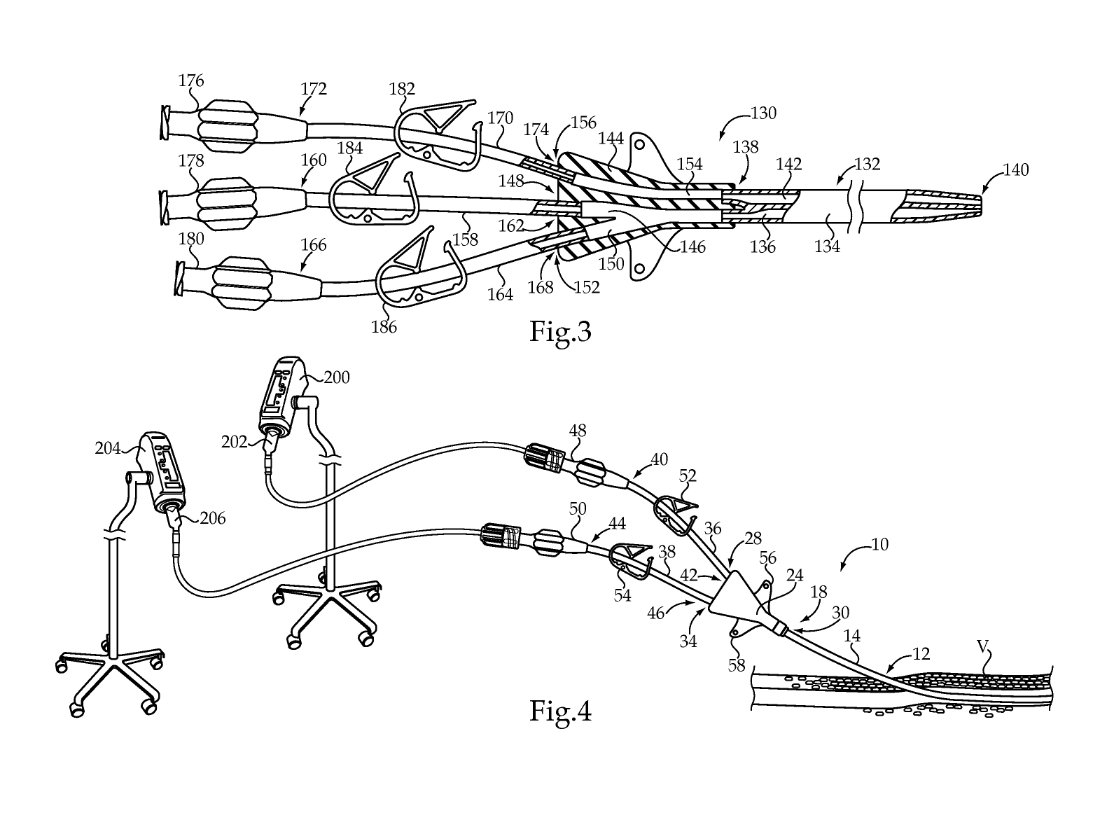

Turning now to FIG. 3, an alternative embodiment of a power injection catheter assembly according to the present disclosure is shown at 130. According to the alternative embodiment, the power injection catheter assembly 130 may include a dual lumen power injection catheter 132. In particular, the dual lumen power injection catheter 132 may include an elongate tubular body 134 defining a power injection lumen 136 extending from an open proximal end 138 to an open distal end 140. The elongate tubular body 134 also defines a non-power injection lumen 142, which may be separate from and not in fluid communication with the power injection lumen 136. The non-power injection lumen 142 may be a conventional lumen that is not necessarily configured to withstand the increased flow rates and pressure of power injection. According to alternative embodiments, however, the additional lumen 142 may be rated for power injection.

The power injection catheter assembly 130 also includes a manifold 144 defining a first power injection channel 146 extending from a first power injection port 148 of the manifold 144 to the power injection lumen 136 of the dual lumen power injection catheter 132 and a second power injection channel 150 extending from a second power injection port 152 to the power injection lumen 136. As shown, the first and second power injection channels 146 and 150 merge within the manifold 144 to direct fluids from the first and second power injection ports 148 and 152 toward the power injection lumen 136. The manifold 144 also defines a non-power injection channel 154 extending from a non-power injection port 156 to the non-power injection lumen 142. According to the exemplary embodiment, the non-power injection channel 154 of the manifold 144 is independent from and does not merge with the power injection channels 146 and 150 of the manifold 144. According to alternative embodiments, however, channel 154 of the manifold may also be rated for power injection, particularly if a catheter having multiple power injection lumens is used.

A first power injection extension tube 158 has a proximal end 160 defining a power injection hub and a distal end 162 attached to the manifold 144 at the first power injection port 148. A second power injection extension tube 164 has a proximal end 166 defining a power injection hub and a distal end 168 attached to the manifold 144 at the second power injection port 152. A non-power injection extension tube 170 has a proximal end 172 defining a non-power injection hub and a distal end 174 attached to the manifold 144 at the non-power injection port 156. The manifold 144 directs fluid flow from the power injection extension tubes 158 and 164 into the power injection lumen 136, and directs fluid flow from the non-power injection extension tube 170 into the non-power injection lumen 142. The power injection catheter assembly 130 of FIG. 3 may also include connectors 176, 178, and 180 and clamps 182, 184, and 186, similar to those described above.

### INDUSTRIAL APPLICABILITY

The present disclosure is generally applicable to medical devices for use in percutaneous vascular procedures, or other procedures involving cavities, ducts, or canals of a patient. More specifically, the present disclosure is applicable to catheter assemblies used for injecting one or more fluids into the vascular system of a patient through a common lumen. Yet further, the present disclosure is applicable to catheter assemblies for power injecting a plurality of different liquids through a common lumen and into the patient vasculature.

Referring generally to FIGS. 1-4, and with particular reference to FIG. 1, a power injection catheter assembly 10 according to the present disclosure may generally include a power injection catheter 12 having an elongate tubular body 14. The elongate tubular body 14 defines a power injection lumen 16 extending from an open proximal end 18 to an open distal end 20. The power injection catheter assembly 10 also includes a manifold 24 attached at the open proximal end 18 of the elongate tubular body 14. The manifold 24 defines a first power injection channel 26 extending from a first power injection port 28 of the manifold 24 to the power injection lumen 16 of the power injection catheter 12 and a second power injection channel 32 extending from a second power injection port 34 of the manifold 24 to the power injection lumen 16.

The power injection catheter assembly 10 may also include two power injection extension tubes 36 and 38. A first power injection extension tube 36 has a proximal end 40 defining a first power injection hub and a distal end 42 attached to the manifold 24 at the first power injection port 28. A second power injection extension tube 38 has a proximal end 44 defining a second power injection hub and a distal end 46 attached to the manifold 24 at the second power injection port 34. As shown, the first power injection channel 26 of the manifold 24 and the second power injection channel 32 of the manifold 24 converge within the manifold 24 to combine fluid flow from the first power injection extension tube 36 and the second power injection extension tube 38. According to alternative embodiments, such as the embodiment of FIG. 2, additional power injection extension tubes and manifold channels may be provided. Additionally or alternatively, a power injection catheter of the present disclosure may define additional lumens, including a non-power injection lumen, as shown in FIG. 3. Clamps may also be provided for selectively closing off fluid flow through a respective power injection extension tube.

With particular reference to FIG. 4, a percutaneous vascular procedure using the power injection catheter assembly 10 of FIG. 1 will be described with reference to a vascular structure V of a patient. Although the catheter assembly 10 may be used in a variety of endovascular procedures, an exemplary percutaneous endovascular procedure may include the injection of contrast media into the vascular structure V. At a first stage of the procedure, a clinician may position a needle, or introducer, through the skin of a patient to gain access to the vascular structure V. Thereafter, the clinician may insert a wire guide through a tube of the introducer and into the vascular structure V. The catheter assembly 10 may be introduced over the wire guide and advanced to a position shown in FIG. 4.

The power injection catheter assembly 10 may also include a first power injector 200 operably connected to the first power injection hub and configured to deliver a first power injectable fluid 202 through the first power injection extension tube 36, the first power injection channel 26, and the power injection lumen 16. A second power injector 204 of the power injection catheter assembly 10 may be operably connected to the second power injection hub and configured to deliver a second power injectable fluid 206, which may be different than the first power injectable fluid 202, through the second power injection extension tube 38, the second power injection channel 32, and the power injection lumen 16. The first power injectable fluid 202 and the second power injectable fluid 206 are combined and directed toward the power injection lumen 16 using the manifold 24. Finally, the combined fluids 202 and 206 are power injected through the power injection lumen 16 and into the patient vessel V.

The power injection catheter assembly disclosed herein provides a means for power injecting multiple power injectable fluids into a common power injection lumen of a catheter. In particular, a manifold of the power injection catheter assembly includes converging channels for combining fluid flow from multiple power injection hubs and directing the combined power injectable fluids through the common power injection lumen.

It should be understood that the above description is intended for illustrative purposes only, and is not intended to limit the scope of the present disclosure in any way. Thus, those skilled in the art will appreciate that other aspects of the disclosure can be obtained from a study of the drawings, the disclosure and the appended claims.

What is claimed is:

1. A power injection catheter assembly, comprising:
a power injection catheter having an elongate tubular body, wherein the elongate tubular body has a proximal end and a distal end and defines a power injection lumen;
a manifold attached at the proximal end of the elongate tubular body, wherein the manifold defines a first power injection channel extending from a first power injection port of the manifold to the power injection lumen, and a second power injection channel extending from a second power injection port of the manifold to the power injection lumen;
a first power injection extension tube having a proximal end defining a first power injection hub and a distal end attached to the manifold at the first power injection port; and
a second power injection extension tube having a proximal end defining a second power injection hub and a distal end attached to the manifold at the second power injection port.

2. The power injection catheter assembly of claim 1, further including:
a first clamp configured to selectively occlude the first power injection extension tube; and
a second clamp configured to selectively occlude the second power injection extension tube.

3. The power injection catheter assembly of claim 1, further including at least one of:
a first power injector operably connected to the first power injection hub and configured to deliver a first power injectable fluid through the first power injection extension tube, the first power injection channel, and the power injection lumen; and
a second power injector operably connected to the second power injection hub and configured to deliver a second power injectable fluid through the second power injection extension tube, the second power injection channel, and the power injection lumen.

4. The power injection catheter assembly of claim 3, wherein at least one of the first power injectable fluid and the second power injectable fluid is a contrast media.

5. The power injection catheter assembly of claim 1, wherein the first power injection channel and the second power injection channel converge within the manifold.

6. The power injection catheter assembly of claim 1, further including a third power injection extension tube having a proximal end defining a third power injection hub and a distal end attached to the manifold at a third power injection port, wherein the manifold further defines a third power injection channel extending from the third power injection port to the power injection lumen.

7. The power injection catheter assembly of claim 1, wherein the elongate tubular body further defines a non-power injection lumen.

8. The power injection catheter assembly of claim 7, further including a non-power injection extension tube having a proximal end defining a non-power injection hub and a distal end attached to the manifold at a non-power injection port, wherein the manifold further defines a non-power injection channel extending from the non-power injection port to the non-power injection lumen.

9. The power injection catheter assembly of claim 1, wherein the power injection catheter is a peripherally inserted central catheter.

10. The power injection catheter assembly of claim 9, wherein the power injection catheter is made from polyurethane.

11. The power injection catheter assembly of claim 9, wherein the power injection catheter has an outer diameter that is equal to or less than 12 French.

12. The power injection catheter assembly of claim 9, wherein the power injection lumen is rated for a flow rate of at least 1 cubic centimeter capacity per second.

13. A method of power injecting fluids using a power injection catheter assembly, wherein the power injection catheter assembly includes: a power injection catheter having an elongate tubular body, wherein the elongate tubular body has a proximal end and a distal end and defines a power injection lumen; a manifold attached at the proximal end of the elongate tubular body, wherein the manifold defines a first power injection channel extending from a first power injection port of the manifold to the power injection lumen and a second power injection channel extending from a second power injection port of the manifold to the power injection lumen; a first power injection extension tube having a proximal end defining a first power injection hub and a distal end attached to the manifold at the first power injection port; and a second power injection extension tube having a proximal end defining a second power injection hub and a distal end attached to the manifold at the second power injection port, the method comprising steps of:
power injecting a first power injectable fluid through the first power injection extension tube and into the manifold;
power injecting a second power injectable fluid through the second power injection extension tube and into the manifold;
directing the first power injectable fluid and the second power injectable fluid toward the power injection lumen using the manifold; and
power injecting the first power injectable fluid and the second power injectable fluid through the power injection lumen and into a patient vessel.

14. The method of claim 13, further including injecting a contrast media through the first power injection extension tube, the first power injection channel, the power injection lumen, and into the patient vessel.

15. The method of claim 13, further including combining the first power injectable fluid and the second power injectable fluid within converging first and second power injection channels of the manifold.

16. The method of claim 13, further including power injecting a third power injectable fluid through a third power injection extension tube connected to the manifold at a third power injection port, a third power injection channel defined by the manifold, the power injection lumen, and into the patient vessel.

17. The method of claim 13, further including injecting a non-power injectable fluid through a non-power injection extension tube connected to the manifold at a non-power injection port, through a non-power injection channel defined by the manifold, through a non-power injection lumen of the power injection catheter, and into the patient vessel.

18. The method of claim 13, further including power injecting the first power injectable fluid and the second power injectable fluid through a peripherally inserted central catheter.

19. The method of claim 18, further including power injecting the first power injectable fluid and the second power injectable fluid through the power injection catheter having an outer diameter that is equal to or less than 12 French.

20. The method of claim 18, further including power injecting the first power injectable fluid and the second power injectable fluid through the power injection catheter at a flow rate of at least 1 cubic centimeter capacity per second.
